(19) United States
(12) Patent Application Publication
(10) Pub. No.: US 2007/0232568 A1
(43) Pub. Date: Oct. 4, 2007

(54) Carbohydrate Composition and Its Use for the Preparation of a Medicament for Treating or Preventing Pulmonary Inflammation or Acute Respiration Distress Syndrome

(75) Inventors: Klaske van Norren, Renkum (NL); Eduard Christiaan van Hoorn, Lelystad (NL); Robert Johan Joseph Hageman, Wageningen (NL); Kelly Jane Lamb, Glasgow (GB)

Correspondence Address: THE WEBB LAW FIRM, P.C., 700 KOPPERS BUILDING, 436 SEVENTH AVENUE, PITTSBURGH, PA, 15219, US

(73) Assignee: N.V. Nutricia, Zoetermeer (NL)

(21) Appl. No.: 10/572,240

(22) PCT Filed: Sep. 20, 2004
(86) PCT No.: PCT/NL04/00649

§ 371 Date: Mar. 14, 2007

(30) Foreign Application Priority Data
Sep. 19, 2003 (EP) 03077972.2

(51) Int. Cl. A61K 31/70; A23L 1/30; A61K 31/7004; A61K 31/715; A61P 11/00; A61K 31/7016; A23L 1/305

(52) U.S. Cl. 514/59; 514/23; 514/54

(57) ABSTRACT

One aspect of the present invention is concerned with a method of treating or preventing pulmonary inflammation as a complication ensuing from physical trauma, bacteraemia or viral inflection, said method comprising enterally administering at least one or more glutathione promoters selected from: −0.3-20 g, preferably 0.5-5 g pyruvate equivalents; −0.1-5 g, preferably 0.2-2 g oxaloacetate equivalents; −0.01-1 g, preferably 0.02-0.5 g lipoic acid equivalents; and at least 20 g of digestible water soluble carbohydrates, in the form of an aqueous liquid composition containing at least 10 g/l of said digestible water soluble carbohydrates. Another aspect of the invention relates to an aqueous liquid composition suitable for enteral administration containing: 2 to 20 wt. % digestible dissolved carbohydrates; two or more glutathione promoters selected from: 0.5 to 50 g/l pyruvate equivalents; 0.05 to 20 g/l oxaloacetate equivalents; 0.05 to 5 g/l cystein equivalents; and at least 45 wt % water.

### TECHNICAL FELD OF THE INVENTION

One aspect of the present invention is concerned with a method of treating or preventing pulmonary inflammation or acute respiratory distress syndrome in a mammal, said method comprising enterally administering to said mammal a liquid nutritional composition.

Another aspect of the invention relates to a liquid nutritional composition for use in said method.

### BACKGROUND OF THE INVENTION

Pulmonary diseases are diseases generally affecting the airways and the lungs and are often accompanied by pulmonary inflammation processes. The airways of the human and animal body consist of a series of tubes and passages that include the throat, the larynx and the trachea. In the chest cavity the trachea divides into the right and left bronchi, or bronchial tubes, that enter the lungs. The branches of the bronchi subsequently become more narrow and form tubes, the bronchioles, that divide into even more narrow tubes, the alveolar ducts. The end of each alveolar duct forms a cluster of thinly walled sacs termed the alveoli.

Pulmonary diseases of an inflamunatory nature such as asthma, emphysemia, acute (or adult) respiratory distress syndrome (ARDS), chronic pulnonary diseases (COPD), pneumonia and bronchitis are common diseases in industrialised countries. These diseases or conditions have recently been increasing at an alarming rate, both in terms of prevalence, morbidity and mortality. In spite of this, their underlying causes still remain poorly understood

ARDS is also known in the medical literature as stiff lung, shock lung, pump lung and congestive atelectasis, and its incidence is 1 out of 100,000 people. ARDS is accumulation within the lung which, in turn, causes the lung to stiffen. The condition is triggered by a variety of processes that injure the lungs. In general ARDS occurs as a medical emergency. It may be caused by a variety of conditions that directly or indirectly cause the blood vessels to “leak” fluid into the lungs. In ARDS, the ability of the lungs to expand is severely decreased and damage to the alveoli and lining (endothelium) of the lung is extensive. The concentration of oxygen in the blood remains very low in spite of high concentrations of supplemental oxygen which are generally administered to a patient. Among the systemic causes of lung injury are trauma, head injury, shock, sepsis, multiple blood transfusions and medications. Pulmonary causes include pulmonary embolism, severe pneumonia, smoke inhalation, radiation, high altitude, near drowning, and more.

ARDS symptoms usually develop within 24 to 48 hours of the occurrence of an injury or illness. It is believed that cigarette smoking may be a risk factor. Among the most common symptoms of ARDS are laboured, rapid breathing, nasal flaring, cyanosis blue skin, lips and nails caused by lack of oxygen to the tissues, breathing difficulty, anxiety, stress and tension. Additional symptoms that may be associated with this disease are joint stiffness and pain and temporarily absent breathing. The diagnosis of ARDS is commonly done by testing for symptomatic signs. A simple chest auscultation or examination with a stethoscope, for example, will reveal abnormal breath sounds which are symptomatic of the condition. Confirmatory tests used in the diagnosis of ARDS include chest X-rays and the measurement of arterial blood gas. In some cases ARDS appears to be associated with other, diseases, such as patients with acute myelogenous leukemia, who developed acute tumour lysis syndrome (ATLS) after treatment with cytosine arabinoside. In general, however, ARDS appears to be associated with traumatic injury, severe blood infections such as sepsis, or other systemic illness, the administration of high dose radiation therapy and chemotherapy, and inflammatory responses which lead to multiple organ failure, and in many cases death.

The death rate from ARDS exceeds 50%. Although many survivors recover normal lung function, some individuals may suffer permanent lung damage, which ranges from mild to severe. Moreover, ARDS patients are often afflicted with complications, such as multiple organ system failures.

Pulmonary inflammation, such as the type typically associated with the disease asthma, is characterised by an increased responsiveness of the trachea and bronchi to various stimuli and manifested by a widespread airway narrowing causing episodic dyspnea, coughing and wheezing and the associated debilitation of the afflicted person. In fact, in severe cases, pulmonary inflammation can result in death.

The primary contributor to the symptoms of asthma is the inflammation of the trachea and bronchial air passages. Accordingly, treatment for asthma has typically included the administration of aerosol formulations including anti-inflammatory steroids. Particularly, it has been found effective to spray anti-inflammatory cortical steroids into the bronchial system prophylactically.

Accordingly, the use of steroidal and hormone-derived compounds in prevention of pulmonary inflammation associated with asthma, has found general acceptance in the art. However, problems are presented by long term use of these compounds such as adrenal insufficiency (which has resulted in fatalities), osteoporosis and other systemic complications.

U.S. Pat. No. 5,998,363 describes a method of treating critically ill patients comprising administering an enteral formulation containing about 2-4 g/l fat, about 50-100 g/l protein hydrolysate, about 160-250 g/l carbohydrate, and water. Examples of carbohydrates mentioned are fructose, maltodextrin, corn syrup and hydrolysed corn starch.

US 2003/0161863 describes a nutritional module for addition to a standard enteral formula at the bed of a patient consisting of a composition containing substances, acting (a) against oxidative stress (e.g. cysteine), (b) for limitation of hypermetabolism/muscle waste (c) for wound healing, (d) for acquired respiratory distress syndrome (ARDS) and other acute inflammatory conditions, (e) for recovery from bone trauma, (f) for reconstituting the gut's microflora. These nutritional modules are meant to be used in the treatment and/or nourishment of critically ill persons.

DE-A 101 51 764 describes a liquid enteral formulation containing per 100 ml:

| | | |
|---|---|---|
| Nitrogen source (protein, oligoprotein and glutamindi- | 6 | g |
| peptide) | | |
| Fat | 2.5 | g |
| Carbohydrates (maltodextrin, polysaccharide, saccharose) | 12 | g |
| Bulking ingredients (soluble and non-soluble) | 1.5 | g |
| Lipoic acid | 400 | mg |
| Vitamin C | 1000 | mg |
| Vitamin E | 200 | mg |
| Selenium | 20 | μg |

| | |
|---|---|
| Minerals, trace elements and further vitamins | conform RDA |

### SUMMARY OF THE INVENTION

The inventors have discovered that there is a correlation between the incidence of pulmonary inflammation following trauma, bacteraemia or viral infection and reduced intake of digestible carbohydrates as a result of fasting during the period shortly before and/or after the occurrence of the trauma, bacteraemia or viral infection. Furthermore, they have unexpectedly found that enteral administration of an aqueous liquid composition containing considerable quantities of digestible water soluble carbohydrates in combination with a glutathione promoter can be particularly effective in maintaining or restoring the resistance of mammals to pulmonary inflammation, especially the resistance to pulmonary inflammation as a complication ensuing from physical trauma, bacteraemia or viral infection. Glutathione promoters that are advantageously employed in accordance with the present invention are pyruvate, oxaloacetate, lipoic acid and biological equivalents of these substances.

### DETAILED DESCRIPTION OF THE INVENTION

Accordingly, one aspect of the invention relates to a method of treating or preventing pulmonary inflammation as a complication ensuing from physical trauma, bacteraemia or viral infection in a mammal, said method comprising enterally administering to said mammal at least one or more glutathione promoters selected from:

0.3-20 g, preferably 0.5-5 g pyruvate equivalents;

0.1-5 g, preferably 0.2-2 g oxaloacetate equivalents;

0.01-1 g, preferably 0.02-0.5 g lipoic acid equivalents;

and at least 20 g of the digestible water soluble carbohydrates, in the form of an aqueous liquid composition containing at least 10 g/l of said digestible water soluble carbohydrates. The indicated amounts refer to the dosages administered during a single administration event or serving and to the amounts of pyruvate, oxaloacetate and/or lipoic acid (or residues of these substances) contained in the amount of liquid composition that is administered during such an event or serving.

The terminology “digestible carbohydrates” as used herein refers to carbohydrates that can either be absorbed as such by the gastrointestinal tract or that can be degraded by the gastrointestinal tract to absorbable components, provided said degradation does not involve fermentative degradation by the intestinal microflora.

The terminology “enterally administering” encompasses oral administration and administration via a tubing that is positioned in the gasto-intestinal tract via different routes in order to allow digestion of the food contents), oral administration being most preferred.

Unless indicated otherwise, the dosages mentioned in this application refer to the amounts delivered during a single serving or single administration event. If the present composition is ingested from a glass or a container, the amount delivered during a single serving or single administration will typically be equal to the content of said glass or container.

In a preferred embodiment, the present aqueous liquid composition is administered in an amount effective to maintain or restore a plasma glutathione to at least physiological level, particularly to a level of at least 15, preferably of at least 20 μM. Even more preferably, the present aqueous liquid composition is administered in an amount effective to maintain or restore a physiological pulmonary glutathione level.

The method according to the present invention is particularly suitable for treating or preventing pulmonary inflammations such as pneumonia, bronchitis, acute (or adult) respiratory distress syndrome (ARDS), and sterile lung infections. Throughout this application the terms acute respiratory distress syndrome and adult respiratory distress syndrome are deemed to be synonyms. In a particularly preferred embodiment, the present method is used to treat or prevent acute respiratory distress syndrome ensuing from physical trauma, bacteraemia or viral infection.

In a particularly preferred embodiment of the invention, the method comprises enterally administering, within 24 hours of the occurrence of a trauma, at least 50 g, more preferably at least 70 g of the digestible water soluble carbohydrates in the form of the aqueous liquid composition. The liquid composition may be administered as a single bolus or, alternatively, it may be administered in two or more doses during the 24 hour period. Preferably, the liquid composition is administered in at least 2 separate doses during the 24 hours period, the administration events preferably being at least 1 hour apart. A particularly suitable protocol comprises administering a sufficient amount of the present liquid composition during the period ranging from 24-8 hours prior to the trauma to deliver at least 40 g of the digestible carbohydrates and to deliver at least 20 g of the digestible carbohydrates during the period of 8-1 hour prior the trauma.

The digestible carbohydrates employed in accordance with the invention may suitably include monosaccharides, disaccharides and polysaccharides. In a particularly preferred embodiment of the present invention the digestible water soluble carbohydrates are largely glucose based. In accordance with this embodiment said digestible water soluble carbohydrates optionally contain saccharides other than glucose in amounts of up to 6%, calculated on the molecular weight of the digestible carbohydrate. Examples of other saccharides that may occur in the digestible glucose based carbohydrates include D-fructose, D-arabinose, D-rhamnose, D-ribose and D-galactose, though preferably these saccharides are not located at the terminal side of the present carbohydrates. The glucose units of oligo—and polysaccharides are preferably predominantly connected via alpha 14 or alpha 1-6 bonds in order to be digestable. The digestible carbohydrates of the invention encompass both linear and branched oligo- and polysaccharides. The number of saccharide units is indicated via a number n. Oligosaccharides have a number of n between 3 and 10; polysaccharides between 11 and 1000 and preferably between 11 and 60.

Preferably, the present liquid composition contains between 30 and 200 g/l of digestible polysaccharides since, in comparison to monosaccharides and disaccharides, polysaccharides are absorbed more slowly. In another preferred embodiment, the composition contains a combination of polysaccharides and mono- and/or disaccharides. More preferably, the digestible carbohydrates comprise between 60-99 wt. % digestible oligo- and/or polysaccharide and between 1-40 wt. % digestible mono- and/or disaccharides. A suitable example of a digestible water soluble oligosaccharide is glucose syrup. Suitable examples of the digestible water soluble polysaccharides include dextrins, maltodextrins, starches, dextran and combinations thereof. Most preferably the water soluble polysaccharide contains at least 50 wt. %, more preferably at least 80 wt. % of polysaccharides selected from the group consisting of dextrin, maltodextrin and combinations thereof, dextrin being most preferred. In a particularly preferred embodiment the digestible carbohydrates include at least 1 wt. % monosaccharide, particularly at least 1 wt. % fructose. Typically, the digestible carbohydrates will contain not more than 20 wt. % fructose in monosaccharide form.

On a daily basis the glutathione promoters are preferably administered in the following amounts:

0.5-50 g, preferably 2-15 g and more preferably 2-5 g pyruvate equivalents;

0.3-20 g, preferably 0.5-10 g oxaloacetate equivalents; and

0.05-5 g lipoic acid equivalents.

The term “pyruvate equivalents” as used in here, encompasses pyruvate as well as salts of pyruvate and precursors of pyruvate, notably precursors that can liberate pyruvate or a pyruvate salt by in vivo conversion, e.g. hydrolysis, of the precursor molecule. Typical examples of pyruvate precursors that can be hydrolysed to produce pyruvate or a pyruvate salt are pyruvate esters.

The terms “oxaloacetate equivalents”, “lipoic acid equivalents” and “cystein equivalents” are defined accordingly. Examples of suitable pyruvate and/or oxolacetate precursors include Krebs cycle intermediates such as citrate, succinate, fumarate and L-malate, citrate and malate being most preferred. Oxaloacetate precursors that are encompassed by the present invention also include the free amino acids aspartate and asparagine (including their salts) as well as oxaloacetate esters. Suitable examples of lipoic acid precursors include lipoic acid esters.

Both pyruvate and oxaloacetate participate in the Krebs cycle and stimulate production of reducing equivalents such as NADH and NADPH. NADPH is required for the intracellular reduction of oxidized glutathione to glutathione by the enzyme glutathione reducase. Thus, enteral co-administration of pyruvate and/or oxaloacetate enhances the positive effect of the present aqueous liquid composition on pulnonary glutathione levels.

It has been suggested that (alpha-)Lipoic acid supplementation increases the de novo synthesis of glutathione. Alpha-lipoic acid, however, does not enhance glutathione synthesis, but instead increases the amount of cysteine, which is a substrate for said synthesis. Han D. et al. (Lipoic acid increases de novo synthesis of cellular glutathione by improving cystine utilization. Biofactors, 1997, 6(3): 321-338) report that lipoic acid reduces cystine and thereby increases the total concentration of the glutathione precursor cysteine. Thus, maintenance or restoration of pulmonary glutathione levels to a physiological level may be facilitated by co-administration of lipoic acid.

Glutathione is a cysteine containing tripeptide, i.e. Glu-Cys-Gly. Cysteine availability is an important factor in the synthesis of glutathione. Thus, also enteral administration of cystein may suitably be employed to help restore or maintain physiological pulmonary glutathione levels. Accordingly, in a preferred embodiment, the present method comprises co-administering 0.1-1 g, more preferably 0.1-0.5 g cystein equivalents. The indicated amounts refer to the amounts of cystein and/or cystein residues that are administered during a single administration event or serving. On a daily basis, cystein equivalents are preferably administered in an amount of 0.1-5 g, more preferably of 0.1-1 g. Typical examples of cystein precursors include proteins, protein hydrolysates and peptides, e.g. whey, whey hydrolysate and cystin.

Another aspect of the present invention concerns an aqueous liquid composition suitable for enteral administration containing:
2 to 20 wt. % digestible dissolved carbohydrates;
two or more glutathione promoters selected from:
   0.5 to 50 g/l, preferably 2-30 g/l pyruvate equivalents;
   0.05 to 20 g/l, preferably 0.5-10 g/l oxaloacetate equivalents;
   0.05 to 5 g/l, preferably 0.24 g/l cysteine equivalents; and
at least 45 wt. % water.

In a particularly preferred embodiment, the liquid composition contains between 0.05 and 5 g/l, more preferably between 0.1 and 4 g/l and most preferably between 0.1 and 2 g/l lipoic acid equivalents. Particularly good results are also obtained if the present composition contains between 0.1 and 30 g/l of pyruvate equivalents, oxaloacetate equivalents or a combination of pyruvate equivalents and oxaloacetate equivalents. In the present method pyruvate and oxaloacetate have a similar biological effect. Because oxaloacetate has slightly more proglutathione activity than pyruvate, the present composition advantageously contains between 0.1 and 10 g/l oxaloacetate equivalents.

As mentioned herein before, cysteine equivalents may suitably be incorporated in the present liquid composition in the from of a protein hydrolysate. Preferably, the present composition contains between 5 and 100 mg/l cysteine equivalents in the form of a protein hydrolysate, preferably in the form of a whey protein hydrolysate.

For patients who find it difficult to swallow or who experience nausea etc., it is important that the digestible carbohydrates can be delivered in concentrated liquid form. Consequently, it is preferred to include the digestible water soluble carbohydrates in a concentration of at least 50 g/l, more preferably of at least 70 g/l and most preferably at least 80 μl.

In order to minimise the risk of regurgitation and also to minimise the residence time in the stomach, it is preferred that the liquid composition contains less than 3 wt. % lipids, more preferably less than 2 wt. % lipids and most preferably less than 1 wt. % lipids. For similar reasons, also the protein level of the present composition is preferably relatively low, especially below 4 wt. %.

The present liquid composition may, for instance, take the form of a solution, a suspension or an emulsion. It is preferred to employ a liquid composition in the form of a solution that contains essentially no undissolved components, e.g. as demonstrated by the fact the liquid composition is clear and transparent.

Yet another aspect of the present invention relates to a composition that can be reconstituted with water to the present aqueous liquid composition. Typically, the reconstitutable composition can take the form of a liquid concentrate, a paste, a powder, granules, tablets etc. Preferably, the reconstitutable composition is a dry product, particularly a dry product with a moisture content of less than 10 wt. %, preferably of less than 7 wt. %.

The invention is further illustrated by means of the following examples.

### EXAMPLES

### Example 1

An aqueous liquid composition to be administered in a serving of 200 ml, comprising per 100 ml:

| | |
|---|---|
| Glucose | 1 g |
| Maltodextrin DE 5 | 10 g |
| Ca-pyruvate | 1 g |

The liquid is to be administered in two servings a day to treat or prevent disorders associated with pulmonary inflammation.

### Example 2

A powder formulation to be reconstituted with water to a serving size of 200 ml:

| | |
|---|---|
| Maltose | 1 g |
| Glucose syrup DE 12 | 10 g |
| Pyvaric acid | 1 g |
| Ca-pyruvate | 0.9 g |
| Whey protein (4% cysteine) | 4 g |

The liquid is to be administered in four servings a day to treat or prevent disorders associated with pulmonary inflammation.

### Example 3

An aqueous liquid composition to be administered in a serving of 200 ml, comprising per 100 ml:

| | |
|---|---|
| Dextrin | 10 g |
| Fructose | 2 g |
| Oxaloacetate | 0.5 g |
| Whey protein (5% cystein) | 3 g |

The liquid is to be administered in three servings a day to treat or prevent disorders associated with pulmonary inflammation.

### Example 4

An aqueous liquid composition to be administered in a serving of 200 ml, comprising per 100 ml:

| | |
|---|---|
| Dextrin | 11.5 g |
| Fructose | 1.3 g |
| Citric acid (oxaloacetate precursor) | 1 g |
| N-acetyl cystein | 3 g |

The liquid is to be administered in four servings a day to treat or prevent disorders associated with pulmonary inflammation.

### Example 5

An aqueous liquid composition to be administered in a serving of 200 ml, comprising per 100 ml:

| | | |
|---|---|---|
| Glucose | 2 | g |
| Glucose syrup DE 19 | 15 | g |
| Oxaloacetate | 100 | mg |
| Lipoic acid | 50 | mg |
| Whey protein (>3% cystein) ^{#} | 2 | g |

^{#} Hydrolysed to a degree of 10%

The liquid is to be administered in two servings a day to treat or prevent disorders associated with pulmonary inflammation.

### Example 6

An aqueous liquid composition to be administered in a serving of 125 ml, comprising per 100 ml:

| | | |
|---|---|---|
| Glucose | 2 | g |
| Glucose syrup DE 29 | 15 | g |
| Oxaloacetate | 100 | mg |
| Lipoic acid | 50 | mg |
| Whey protein (>3% cystein) ^{#} | 4 | g |

^{#} Hydrolysed to a degree of 10%

The liquid is to be administered in three servings a day to treat or prevent disorders associated with pulmonary inflammation.

### Example 7

An aqueous liquid composition to be administered in a serving of 500 ml, comprising per 100 ml:

| | | |
|---|---|---|
| Glucose | 2 | g |
| Glucose syrup DE 32 | 15 | g |
| Oxaloacetate | 50 | mg |
| Lipoic acid | 25 | mg |
| Whey protein (>3% cystein) ^{#} | 2 | g |
| Casein | 3.5 | g |

^{#} Hydrolysed to a degree of 10%

The liquid is to be administered by tube feeding in two to four servings a day to treat or prevent disorders associated with pulmonary inflammation.

### Example 8

Rat studies were carried out to determine the effect of pre-operative feeding of carbohydrates on post-operative pulmonary inflammation rate.

Experimental Set Up Surgery:
The rats were allowed ad libitum autoclaved chow feeding until 16 hours before the operation (Table I). The intervention group received 113 g of dextrin, 12.7 μl fructose plus an isotonic mix of salts and citric acid in drinking water, starting 5 days before the operation and continued until the day of operation. Ad libitum water served as control. The operation was started by performing a laparotomy followed by clamping of the superior mesenteric artery for 60 minutes followed by a reperfusion period of 180 minutes. After this period blood was collected via cardiac puncture. Subsequently, animals were sacrificed, organs were collected and immediately frozen in liquid nitrogen.

Pulmonary Neutrophil Infiltration Rate:
A 1.25% lung homogenate was prepared, with 20 mM phosphate buffer pH=7.4. It was centrifuged at 3600×g at 4° C. for 15 minutes. The pellet was re-homogenized in 1500 μl 50 mM phosphate buffer (pH=6.0) containing 0.5% hexadecyltrimethyl ammonium bromide (HETAB) and 10 mM EDTA. To 50 μl of this sample 450 μl of buffer A (37° C.) was added. (Buffer A: 12 ml water, 1.6 ml of 1M phosphate buffer pH=5.4, 3.2 ml of 0.3 g 3,3′, 5,5′-tetramethylbensidine and 1 ml of 10 g Hetab in 100 ml milli Q™) MPO activity was measured kinetically, at 655 nm.

Determination of Reduced and Oxidized Glutathion in Tissues
Approximately 50 mg tissue (5 mg) (−20° C.) was cut on an ice-cold glass dish then transferred to a 15 ml falcon tube and weighed on an analytical balance. To 50 mg of tissue exactly 1000 μl of 0.4 M HClO4 was added and processed as described by van Hoorn et al [van Hoorn, 2003#4916] and centifuged at 13000 rpm and +4° C. 50 μl of supernatant was diluted three times with 0.5 M phosphate-EDTA buffer pH7.5.
For the measurement of oxidized glutathione (GSSG), 40 μl of tissue extract was added to 5 μl 2-vinylpyridine in a 96 well plate and allowed to react for one hour at room temperature. Then 20 μl 5,5′-dithio-bis(2-nitrobenzoic acid) (DTNB) was added. This was directly followed by addition of 40 μl of glutathione reductase (5 mg/ml). After mixing the plate, the reaction was started by addition of 100 μl NADPH (0.333 mg/ml). The kinetics of this reaction was measured immediately after addition of NADPH, every 15 seconds, for a total of 15 times, at 405 nm. Total glutathione (reduced and oxidized) was measured in the same way, only the 2-vinylpyridine was omitted. Reduced glutathione (GSH) concentration was calculated by subtracting twice the estimate for GSSG from total glutathione measured as one mole of GSSG is reduced by the glutathione reductase to produce 2 moles of reduced GSH.

Results:

[image]

Neutrophil Infiltration of the Lung
A. Pre-operative carbohydrate fed rats showed a significantly decreased (P<0.02) neutrophil infiltration (expressed as myeloperoxidase activity) when compared to IR fasted rats.
B. Carbohydrate-supplemented rats showed a significant increase in pulmonary GSH concentration when compared to IR fasted animals (P=0.014)

### Example 9

HepG2 cells, a human hepatocarcinoma cell line, were obtained from ATCC. These were maintained in MEM supplemented with 10% FCS; 1% NEAA; 1% penicillin/streptomycin mixture. Cells were seeded primarily at a density of approximately 1-2×10⁶ cells and were split and transferred to new flasks when showing 70-90% confluency.

96-well microtitre plates (ex Micronic, Leylstad, NL.), containing 0.35×10⁶ cells per well were incubated for 24 hours at 37° C.; 5% CO₂. Media was removed and 100 μl cell media containing increasing pyruvate or oxaloacte concentrations was added to each well. Cells were incubated for a further 24 hours. After the 24 hours, media was removed, wells were washed twice with PBS and cells were lysed by the addition of 100 μl of demineralised H₂O per well followed by incubation for 30 mins at 37° C.; 5% CO₂.

Glutathione concentrations were measured spectrophotometrically based on the method of Tietze et al. (Anal Bioch (1969) 27, 502-522). The reaction was measured at 405 nm using a kinetic assay protocol measuring A 405 nm every 15 seconds, 15 times, a total reaction time of 3 minutes 45 seconds. Mix time was 2 seconds. 0.1M Phosphate-EDTA buffer was used as the assay diluent. Cell lysates were diluted by a factor of two to ensure that values were within assay parameters.

The glutathione concentrations concentrations measured are depicted in FIGS. 1 and 2 as a function of the applied oxaloacetate and pyruvate concentrations. The results show that both oxaloacetate and pyruvate are capable of increasing glutathione levels in HepG2 cells.

[image]

[image]

1-14. (canceled)

15. A method of treating or preventing pulmonary inflammation as a complication ensuing from physical trauma, bacteraemia or viral infection in a mammal, comprising enterally administering an aqueous liquid composition comprising digestible water soluble carbohydrates and one or more glutathione promoters, said glutathione promoters selected from:
0.3-20 g pyruvate equivalents, said pyruvate equivalents selected from pyruvate, pyruvate salts and pyruvate esters;
0.1-5 g oxaloacetate, oxaloacetate salts and/or oxaloacetate esters; and
a combination of (a) 0.01-1 g lipoic acid equivalents, said lipoic acid equivalents selected from lipoic acid, lipoic acid salts and lipoic acid esters; and (b) 0.1-5 g oxaloacetate equivalent, said oxaloacetate equivalents selected from oxaloacetate, oxaloacetate salts, oxaloacetate esters, aspartate, aspartate salts, asparagine and asparagine salts;
and at least 20 g of the digestible water soluble carbohydrates, in the form of the aqueous liquid composition containing at least 10 g/1 of said digestible water soluble carbohydrates.

16. The method according to claim 15, wherein 0.5-5 g of pyruvate equivalents are administered.

17. The method according to claim 15, wherein 0.2-2 g oxalacetate, oxaloacetate salts and/or oxaloacetate esters are administered.

18. The method according to claim 15, wherein the pulmonary inflammation is acute respiratory distress syndrome.

19. The method according to claim 15, wherein the liquid composition is administered to a mammal suffering from trauma, said administration taking place within 24 hours of the occurrence of the trauma.

20. The method according to claim 15, wherein the liquid composition contains between 30 and 200 g/l of digestible polysaccharides.

21. The method according to claim 15, wherein the digestible water soluble carbohydrates are selected from the group consisting of dextrins, maltodextrins, starches and dextran.

22. The method according to claim 15, further comprising co-administering 0.1-1 g cystein and/or cystein residues.

23. The method according to claim 15, further comprising co-administering 0.1-0.5 g cystein and/or cystein residues.

24. An aqueous liquid composition suitable for enteral administration containing:
2 to 20 wt. % digestible dissolved carbohydrates;
two or more glutathione promoters selected from:
0.5 to 50 g/l pyruvate equivalents, said pyruvate equivalents selected from pyruvate, pyruvate salts and pyruvate esters;
0.05 to 20 g/l oxaloacetate, oxaloacetate salts and/or oxaloacetate esters;
0.05 to 5 g/l cystein and/or cystein residues; and
at least 45 wt. % water.

25. An aqueous liquid composition suitable for enteral administration containing:
2 to 20 wt. % digestible dissolved carbohydrates;
two or more glutathione promoters selected from:
2-30 g/l pyruvate equivalents, said pyruvate equivalents selected from pyruvate, pyruvate salts and pyruvate esters;
0.5 to 10 g/l oxaloacetate, oxaloacetate salts and/or oxaloacetate esters;
0.2 to 4 g/l cystein and/or cystein residues; and
at least 45 wt. % water.

26. The liquid composition according to claim 24, wherein the composition further contains between 0.05 and 5 g/l lipoic acid equivalents, said lipoic acid equivalents selected from the group consisting of lipoic acid, lipoic acid salts and lipoic acid esters.

27. The liquid composition according to claim 24, wherein the composition further contains between 0.1 and 2 g/l lipoic acid equivalents, said lipoic acid equivalents selected from the group consisting of lipoic acid, lipoic acid salts and lipoic acid esters.

28. The liquid composition according to claim 24, wherein the composition contains between 0.1 and 30 g/l of:
a) pyruvate equivalents, said pyruvate equivalents selected from the group consisting of pyruvate, pyruvate salts and pyruvate esters;
b) oxaloacetate, oxaloacetate salts and/or oxaloacetate esters; or
c) a combination of (a) and (b).

29. The liquid composition according to claim 28, wherein the composition contains between 0.1 and 10 g/l oxaloacetate, oxaloacetate salts and/or oxaloacetate esters.

30. The liquid composition according to claim 24, wherein the composition contains between 5 and 100 mg/l cystein and/or cystein residues in the form of a protein hydrolysate.

31. The liquid composition according to claim 30, wherein the protein hydrolysate is in the form of a whey protein hydrolysate.

32. The liquid composition according to claim 24, wherein the liquid composition is a clear aqueous solution.

33. A composition that can be reconstituted with water to a liquid composition according to claim 24.
